(19) United States
(12) Patent Application Publication
(10) Pub. No.: US 2022/0095695 A1
(43) Pub. Date: Mar. 31, 2022

(54) INHALATION DEVICE WITH CYLINDRICAL ROTATABLE DIAL FOR INPUT OF A TARGET AMOUNT OF INHALED SUBSTANCE

(71) Applicant: INDOSE INC, Woodland Hills, CA (US)

(72) Inventors: Ari FREEMAN, Woodland Hills, CA (US); Daniel FREEMAN, Woodland Hills, CA (US); Jacqueline FREEMAN, Woodland Hills, CA (US); Benzi RONEN, Woodland Hills, CA (US); John DALE, Woodland Hills, CA (US); George BERNOTA, Woodland Hills, CA (US); Mike DAUGHTERY, Woodland Hills, CA (US); Kelly MCQUINN, Woodland Hills, CA (US)

(73) Assignee: INDOSE INC, Woodland Hills, CA (US)

(21) Appl. No.: 17/545,666

(22) Filed: Dec. 8, 2021

Related U.S. Application Data
(63) Continuation of application No. 16/707,854, filed on Dec. 9, 2019, now abandoned.
Application No. 16/707,854 is a continuation-in-part of application No. 16/515,860, filed on Jul. 18, 2019, now abandoned.
Application No. 16/515,860 is a continuation-in-part of application No. 15/244,518, filed on Aug. 23, 2016, now Pat. No. 10,595,562.
(60) Provisional application No. 62/388,066, filed on Jan. 13, 2016.
(60) Provisional application No. 62/386,614, filed on Dec. 7, 2015.
(60) Provisional application No. 62/386,615, filed on Dec. 7, 2015.

(51) Int. Cl. A24F 40/57; A24F 40/60; A61M 15/06; A24F 40/51; A61M 15/00

(52) U.S. Cl. CPC A24F 40/57; A24F 40/60; A61M 15/06; A24F 40/65; A61M 15/0083; A61M 15/0066; A61M 15/0071; A24F 40/51

(57) ABSTRACT

An inhalation device for e.g., nicotine products which receives a user input regulating the amount of nicotine the user wants to receive. Once the selected amount has been delivered to the user, the device shuts off its atomizer to conclude operation.

### CROSS-REFERENCE TO RELATED APPLICATIONS

This is a continuation of U.S. application Ser. No. 16/707,854 filed Dec. 9, 2019, which is a continuation in part of U.S. application Ser. No. 16/515,860, filed Jul. 18, 2019, which is a continuation in part of U.S. application Ser. No. 15/244,518, filed Aug. 23, 2016, which claims the benefit of the filing dates of U.S. Provisional Patent Application Nos. 62/386,614 and 62/386,615, filed Dec. 7, 2015, and 62/388,066, filed Jan. 13, 2016. The above-named applications are incorporated by reference herein in their entireties.

### BACKGROUND

Inhaling devices such as vaporizers, vaporizing pens, and vaporizing machines are used to vaporize substances such as tobaccos, oils, liquids, medical drugs, and plant-based oils and herbs. Once vaporized, these substances are then inhaled by consumers. Using vaporizers or vape pens has become a popular method for inhaling tobacco-based and herbal vapor products. The most common method of using a vaporizer involves attaching a cartridge to a battery unit. These cartridges contain the oil reservoir (or other liquid/wax/herbal substances), an atomizer unit, a mouthpiece and a battery connector. The oil reservoir contains the substance that is heated and vaporized. The atomizer gets electric power from the battery using the battery connector and heats to a specific temperature. The battery connector is most commonly made via a 510 thread to the battery unit. Thereby, these types of cartridges are often referred to as 510 cartridges or carts. Other types of connections exist as well, such as; magnetic or snap in connections.

Such inhaling devices have health benefits over traditional smoking methods. But inhaling the vapor can have negative effects on the body depending on the substance, such as nicotine. Inhaling devices have become more popular with consumers, but pose problems.

For example, while vaporizers can be safer than traditional smoking methods, it is difficult to meter the amount of vaporized substance that is being inhaled. So a user of an inhalation device that vaporizes nicotine may actually consume more nicotine than had the user smoked cigarettes or cigars.

There are multiple factors that affect the quantity of drug that is inhaled. These factors include the drug concentration of the vaporized substance, the amount of vapor inhaled, the duration of inhalation, variations between inhalation devices, and variation and inconsistency in the functionality of the device.

Another issue is that the inhaled substances may have different effects on different users depending on various factors. To optimize a user's experience, it is necessary to track the quantity inhaled/taken over time and track the resulting effect it has on that user. This can be a tedious and demanding task. Typical users may not keep track of each dose and record the experience.

### SUMMARY

According to an aspect of the disclosure, an inhalation device may include a dose selection component configured to receive, via a user input, a dose selection of a substance to be vaporized by the inhalation device, and a controller configured to determine that a dose corresponding to the dose selection has been generated by the inhalation device, and control a component of the inhalation device based on determining that the dose corresponding to the dose selection has been generated by the inhalation device.

According to an aspect of the disclosure, a method includes receiving, via a dose selection component of an inhalation device, a user input that identifies a dose selection of a substance to be vaporized by the inhalation device, determining that a dose corresponding to the dose selection has been generated by the inhalation device, and controlling a component of the inhalation device based on determining that the dose corresponding to the dose selection has been generated by the inhalation device.

### BRIEF DESCRIPTION OF THE DRAWINGS

FIG. 1 is a diagram of an inhalation device;

FIG. 1A is a diagram of a portion of an inhalation device;

FIG. 1B is another diagram of a portion of an inhalation device;

FIG. 2 is another diagram of an inhalation device;

FIG. 3 is another diagram of an inhalation device;

FIG. 4 is another diagram of an inhalation device;

FIG. 5 is another diagram of an inhalation device;

FIG. 6A is another diagram of an inhalation device;

FIG. 6B is a diagram of a inhalation device and a battery unit;

FIG. 6C is a diagram of the inhalation device connected to the battery unit;

FIG. 7 is a diagram of a battery cartridge;

FIG. 8 is another diagram of an inhalation device;

FIG. 9 is a diagram of a process for controlling a component of a vaporizer based on determining that a dose corresponding to a dose selection has been generated; and

FIG. 10 is another diagram of an inhalation device.

### DETAILED DESCRIPTION

The present disclosure allows consumers to control the amount of a vapor and thereby the amount of a substance, such as a medicant or drug, that they are inhaling from a cartridge. More specifically, consumers can choose the number of doses they inhale by rotating an incremented dial device to a desired level. Once the desired level of vapor is inhaled, the device automatically shuts off the atomizer and no more vapor is produced. The device notifies to the consumer that they have achieved their desired dose using a built in indicator light.

As described in more detail elsewhere herein, the present disclosure provides a cartridge that attaches to a battery unit, and that includes a reservoir tank, an atomizer or heater, a dosage or amount selector, a light indicator, a battery connector, and a mouthpiece. The device may connect to a battery unit. The consumer selects their desired dosage level using the dose dial, and begins to inhale from the mouthpiece. The indicator light turns on indicating that vapor production has begun. The device turns on the atomizer and begins creating vapor. The device may use an airflow sensor to calculate airflow rate. The device may adjust the amount of power being delivered to the atomizer's heater to adjust temperature according to the airflow rate. The device uses the measurements or estimates of airflow rate and power to calculate the amount of time the heater will stay on to allow the consumer to inhale their desired amount. Once the targeted amount of time has been completed, the atomizer turns off. The light indicator turns off indicating to the consumer that their desired dose has been completed.

The dial device as disclosed in these embodiments may also operate as a security device. In this implementation, the dial is used for passcode entry when the user first picks up the inhaler. A sequence of dial settings within a limited amount of time can serve as the passcode, to permit the atomizer of the inhaler to be powered up.

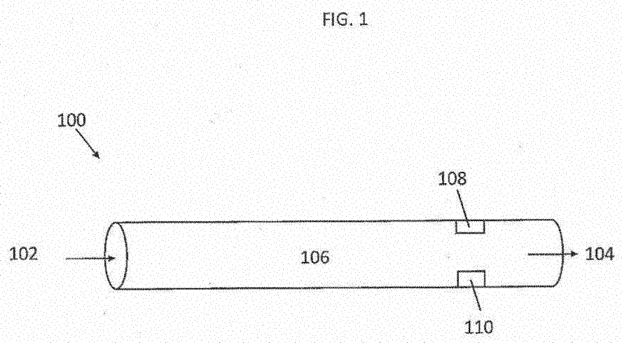

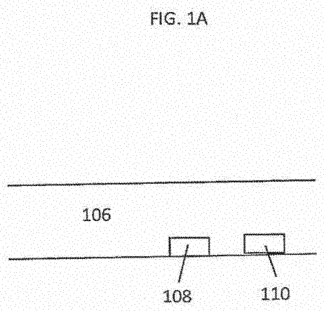

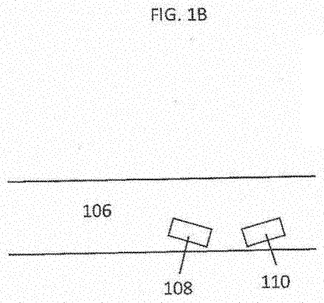

FIG. 1 illustrates an inhalation device 100 for inhaling a vaporized substance. The inhalation device 100 includes a first opening 102 and a second opening 104. In between the two openings is a channel 106. When a user inhales using the inhalation device 100, air flows into the first opening 102 and in the inhalation device 100, vaporized substance is created by a heating element (not shown), and a mixture of air and vapor flows through the channel 106 to the second opening 104 and ultimately to the user.

The inhalation device 100 also includes a sensor 108 and a signal 110. The sensor 108 and signal 110 are positioned across from each other in the channel 106. The sensor 108 senses the vapor amount. For example, the sensor 108 can sense the concentration of vapor. The sensor 108 senses the intensity of the signal emitted by the signal 110. If the sensor 108 senses a high signal output, this indicates that the amount of vapor is low, and the vapor/air mixture is dominated by air. Likewise, if the sensor 108 senses a low signal output, this indicates that the vapor/air mixture is dominated by vapor.

Data from the sensor 108 can assist the inhalation device 100 in providing information about vapor concentration to the user. For example, if the sensor senses a 5% drop in intensity from the signal 110, that could correlate to a mixture of vapor/air that is 60% vapor.

Knowing the relative concentration of the vapor can assist the inhalation device 100 in providing additional information to the user. For example, if a user inhales using the inhalation device 100 and the sensor 108 senses a high output, this may indicate that the concentration is less than expected. The inhalation device 100 could include an additional indicator to inform the user that the inhalation device 100 is not producing the expected amount of vapor. The sensor 108 can be any suitable sensor that senses light including without limitation, a photosensor, photodetector, optocell, optoresistor, optotransistor, optodiode, and/or solar cell. The signal 110 can be any suitable device that produces light, such as an LED. The signal could also emit ultraviolet light. In other words, the signal 110 can produce a wide range of wavelengths of light and the sensor 108 detects those wavelengths of light. The inhalation device 100 can optionally use filters in order to target a specific wavelength of light to optimally detect vapor intensity. Additionally, the reservoir can be configured to connect wirelessly to a mobile device (such as a smartphone), either directly or via the processor 604. This would allow the user to track their drug usage using a mobile application. This wireless connection could take many forms, including; Bluetooth, Wi-Fi, cellular, near field radio, transponder and others.

In FIG. 1, the sensor 108 is positioned across from the signal 110. The sensor 108 and the signal 110 can also be positioned in alternative arrangements without departing from the scope of this disclosure. For example, in FIG. 1A the sensor 108 and the signal 110 are positioned next to each other in the channel 106. In another embodiment, shown in FIG. 1B, the sensor 108 and the signal 110 are positioned next to each other at an angle in the channel 106. The arrangements of the sensor 108 and the signal 110 in FIGS. 1A and 1B use concepts of backscatter and fluorescence.

In backscatter, the vapor passing through the channel 106 can “reflect” light back from the perspective of the sensor. In this scenario, the vapor particle size would determine the “reflection” properties and angle of refection. In florescence, the light may get absorbed by the vapor particles and a new light may be generated. The new light would then be picked up by the sensor. The light and sensor may be set up facing the same direction (in parallel) towards the channel 106. Other alternative positions of sensor 108 and signal 110 known to persons of ordinary skill in the art whereby the flow of vaporized substance affects the signal received by the sensor from the light produced by the light signal device is intended to fall within the scope of this disclosure. For example, the sensor 108 and the signal 110 may be next to each other but one of the sensor 108 and the signal 110 may also be positioned at an angle.

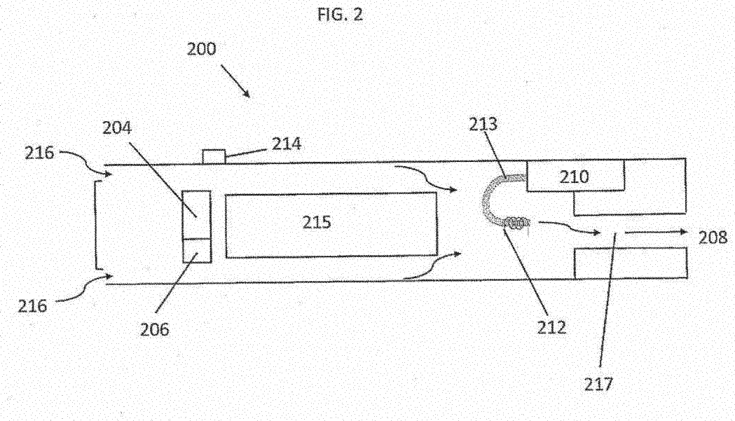

FIG. 2 shows an inhalation device 200. The inhalation device includes a processor 204 and a timer 206. In this embodiment, the inhalation device 200 includes an inlet 216, an outlet 208, a reservoir 210, a heating element 212, and a wick 213. The inhalation device 200 also includes an indicator 214 and a battery 215. The reservoir 210 stores the substance in unvaporized form, and the heating element 212 heats the unvaporized substance from the reservoir 210 via the wick 213 to create a vaporized substance, which is then inhaled by the user through the outlet 208. The inhalation device 200 also includes a channel 217 through which the vaporized substance produced by the heating element 212 and air will flow to the outlet 208 when a user inhales.

The inhalation device 200 uses the processor 204 and the timer 206 to provide metering information to the user. More specifically, the processor 204 controls the timer 206 such that when a user inhales using the inhalation device 200, the processor 204 will start the timer 206 as well as the heating element 212 to begin vaporizing the substance. After the timer 206 has reached a particular value, a particular amount of the vaporized element will have been produced, and the processor 204 will shut off the heating element 212. Alternatively, the processor 204 will not shut off the heating element 212, but rather will send a signal to the indicator 214 that the particular amount of the vaporized substance has been consumed.

For example, if the heating element produces 1 mg/second, and the particular amount is 3 mg, the processor will turn on the heating element 212 when a user inhales, and the processor will turn off the heating element when the timer reaches 3 seconds. After the timer reaches 3 seconds, the processor will send a signal to the indicator 214, which will then indicate that the particular amount has been consumed. The indicator 214 can be an audio signal, visual signal, visual display, or a vibration. The indicator 214 could also be a transmitter that sends a signal to an external device such as a smart phone, tablet, or computer indicating that a particular amount has been consumed.

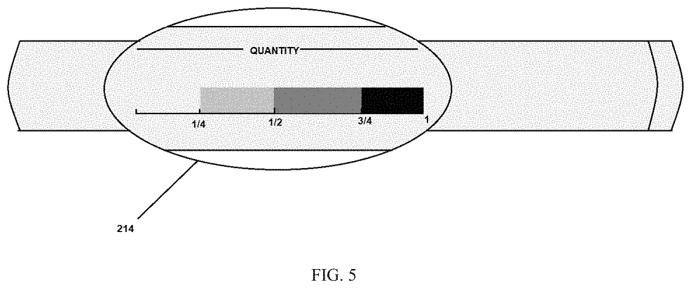

Alternatively, the indicator 214 could display what amount the user has consumed. As shown in FIG. 5, as a visual indicator to the user, the indicator 214 may include a progressive meter indicator. This could take the form of a sequence of lights, possibly LED lights, which indicate the progression of the amount consumed by the user. For example, there could be a sequence of four LED lights on the vaporizer indicating when 25%, 50%, 75% and the full amount has been taken. When the full amount has been taken, the lights might be programmed to indicate to the user that the full amount has been reached by flashing. The progressive meter indicator could take other forms, like a mechanical indicator, a dial, a screen display, or a sound sequence. The progressive meter indicator may continue to meter and indicate the user beyond one cycle. For example, after a full amount has been taken the indicator will turn all lights off and begin turning on each light again as the user consumes.

In the above example, in which a particular amount is set at 3 mg and the heating element 212 produces 1 mg/second of vapor, 3 mg will be delivered to a user who inhales for 3 seconds. In the event that the user cannot inhale long enough to consume a single dose in a single inhalation, the inhalation device 200 is configured to keep a session open, with a session being defined as a particular time within which a user can consume the particular amount. A session in this case could be set to 10 seconds. In this open session configuration, the inhalation device 200 can stop producing vapor when the user stops inhaling and start producing vapor when the user inhales again. When the sum of the user's inhalations amounts to consumption of 3 mg, the processor will send a signal to the indicator 214. Determining when the user stops inhaling can be achieved by using a pressure sensor. Where the pressure drops below a threshold, the heating element will stop. And when the pressure goes above the threshold, the heating element will resume. Alternatively, instead of time-based, a session can be vapor-based, where the inhalation device 200 keeps a session open until a certain quantity of vapor is produced.

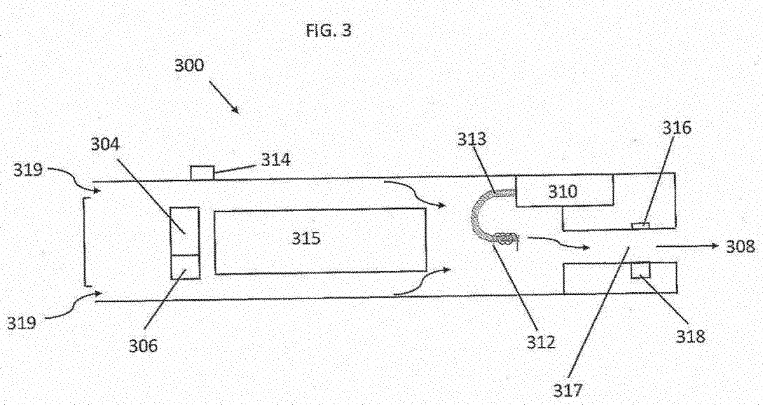

FIG. 3 shows an inhalation device 300 according to another embodiment. The inhalation device includes a processor 304 and a timer 306. In this embodiment, the inhalation device 300 includes an inlet 319, an outlet 308, a reservoir 310, a heating element 312, and a wick 313. The inhalation device 300 also includes an indicator 314 and a battery 315. The reservoir 310 stores the substance in unvaporized form, and the heating element 312 heats the unvaporized substance from the reservoir 310 via the wick 313 to create a vaporized substance, which is then inhaled by the user through the outlet 308. The inhalation device 300 also includes a channel 317 through which the vaporized substance produced by the heating element 312 and air will flow to the outlet 308 when a user inhales.

The inhalation device 300 further includes an indicator 314 that will indicate to the user when a particular amount of the vaporized substance has been consumed. The indicator 314 can be an audio signal, visual signal, visual display, or a vibration. The indicator 314 could also be a transmitter that sends a signal to an external device such as a smart phone, tablet, or computer indicating that a dose has been consumed. Alternatively, the indicator 314 could display what dose the user has consumed.

The inhalation device 300 can also include a sensor 316 and a signal 318, such as an LED that produces a wide range of light wavelengths. The signal could also be one that produces ultraviolet light. The sensor 316 and signal 318 are positioned across from each other in the channel 317. The sensor 316 senses the concentration of the vapor. For example, the sensor 316 can be an optical sensor that senses the intensity of the light produced by the signal 318. If the sensor 316 senses a high output, this indicates that the vapor concentration is low, and the vapor/air mixture is mostly, if not all, air. If the sensor 316 senses a low output, this indicates that the vapor concentration is high. The processor 304 records information from the sensor 316. The sensor 316 can assist the inhalation device 100 in providing information about vapor concentration to the user. For example, if the sensor senses a 5% drop in intensity from the signal 110, that could correlate to a mixture of vapor/air that is 60% vapor.

The processor 304 uses data from the sensor 316 to calculate when a particular amount of the vaporized substance has been produced. This is useful where the substance is viscous. In such viscous substances the amount of vapor produced for a given time can vary. In the embodiment of FIG. 3, when a user inhales using the inhalation device 300, the processor 304 will turn on the heating element 312. The sensor 316 will sense in real time (as a non-limiting example, every 0.1 seconds) the intensity of the light from the signal 318. Using the data from the sensor 316, the processor 304 can determine when a particular amount has been produced.

For example, if a particular amount to be consumed is 3 mg and the heating element 312 vaporizes for example 1 mg per second, then theoretically the 3 mg should be produced in 3 seconds. In practice, however, it may take longer for the inhalation device 300 to vaporize 3 mg. This may due to factors such as the time it takes the heating element 312 to heat up and the consistency of the drug released from the reservoir 310 to the wick 313. So for example, when a user begins to inhale, the first ten readings of the sensor 316 in the first second (e.g., one reading every 0.1 seconds) may indicate that the vapor produced over the first second is 50% of the expected production. This percentage can be thought of as a vapor factor. The processor 304 will take this vapor factor into account to determine when 3 mg is consumed by the user. In other words, the processor 304 will collect the data from the sensor 316 (e.g., every 0.1 seconds) on the vapor factor to determine when 3 mg has been consumed by the user. For a given time, the processor 304 will multiply the time (e.g., 0.1 seconds) by the vapor factor at that time, and will add each of these products to determine when a particular amount has been consumed. For example, if in the first second of inhalation, 50% of vapor is produced, and assuming 100% of vapor is produced after 1 second, the processor will able to determine that 3 mg has been consumed in 3.5 seconds.

In the above example, the processor 304 is capable of acquiring data from the sensor 316 and also included information on how much a particular amount of substance is expected to be produced per unit of time. The processor 304 can store additional vapor characteristics of the substance. For example, the processor 304 can store the time it takes for the heating element 312 to heat to the temperature at which it vaporizes the substance. The processor 304 can also store the heating and temperature variations during different inhalation profiles. For example, if a user inhales at a high rate, the air flowing through the inlet 319 and into the inhalation device 300 can cool the heating element 312. The processor 304 can store information on different rates of inhalation to adjust, for example, the temperature of the heating element 312. The processor 304 can also store information on the flow of drug from the reservoir 310 to the wick 313, the concentration of the substance within a given volume, and the vaporization rates of the substance at different temperatures of the heating element 312. The processor 304 as well as the processors discussed herein can be standard integrated circuit (IC) chips made by IC manufacturers such as Texas Instruments.

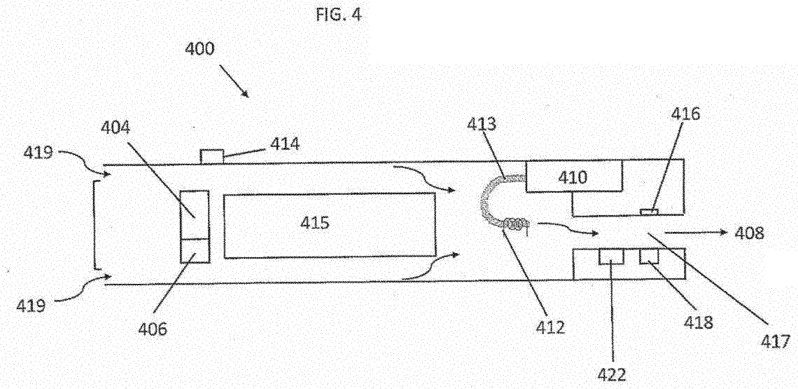

FIG. 4 illustrates another inhalation device 400 according to another embodiment of the disclosure. The inhalation device 400 includes a processor 404 and a timer 406. In this embodiment, the inhalation device 400 includes an inlet 419, an outlet 408, a reservoir 410, a heating element 412, and a wick 413. The inhalation device 400 further includes an indicator 414 for informing a user when a dose of the substance has been inhaled and a battery 415. The inhalation device 400 also includes a channel 417 through which air and the vaporized substance produced by the heating element 412 flow to the outlet 408 when a user inhales.

The inhalation device 400 also includes a sensor 416 and a signal 418, such as an LED that produces a wide range of light wavelengths. The signal could also be one that produces ultraviolet light. The sensor 416 and signal 418 are positioned across from each other in the channel 417. The sensor 416 senses the concentration of the vapor. For example, the sensor 416 can be an optical sensor that senses the intensity of the light produced by the signal 418 at wavelengths that would include, but not be limited to, visible light and ultraviolet light.

The inhalation device 400 further includes a volume flow sensor 422. The volume flow sensor 422 can be any suitable airflow sensor including, but not limited to, any combination or stand-alone of the following: a pressure sensor, a propeller, a microphone or a piezoelectric sensor. The volume flow sensor 422 is used to measure the velocity at which the mixture of vapor and air flow through the channel 417. So for example, if the volume flow sensor 422 is a propeller, the propeller would be installed in the channel 417 and would spin according to velocity of the vapor/air mixture. The frequency of revolutions can be measured and used to calculate the velocity of the mixture. If the sensor is a microphone, the microphone can be setup in the channel 417 to listen to the noise of the vapor/air mixture passing through the channel. A correlation can be made between the sound intensity and/or frequency to the rate of flow of the mixture. Optionally, the volume flow sensor 422 can be placed between the inlet 419 and the processor 404 such that it detects the air flow rate going through the inhalation device 400 when a user inhales. If desired, to obtain a particularly economical device, the airflow sensor could be replaced with an airflow restrictor, such that the airflow can be estimated within a narrow accuracy band, and thereby essentially rendered as a constant.

The volume flow sensor 422 can be used to adjust the intensity of the heating element 412. The temperature of the heating element can affect the amount of the substance that is vaporized. The volume flow sensor 422 is able to sense how intensely a user inhales (i.e., senses the volume per unit time of an inhalation). The processor 404 can acquire this data and adjust the intensity of the heating element by adjusting the voltage of the heating element.

The volume flow sensor 422 and the adjustment of the heating element 412 is useful in a non-limiting situation where the user desires to consume a dose more quickly. So for example, if the inhalation device 400 is set up so that the heating element produces 1 mg/second of vapor and a dose is 3 mg, a user that inhales at a high volume per unit time can consume the entire dose quicker than 3 seconds. In this scenario, the volume flow sensor 422 will be able to sense the higher velocity of the vapor/air mixture, and the processor can increase the intensity of the heating element such that it produces more vapor. The processor 404 can adjust the intensity of the heating element 412 in real time based on data from the volume flow sensor 422. So if a user does not inhale intensely, the volume flow sensor 422 will detect the decreased flow rate and the processor can then lower the intensity of the heating element 412.

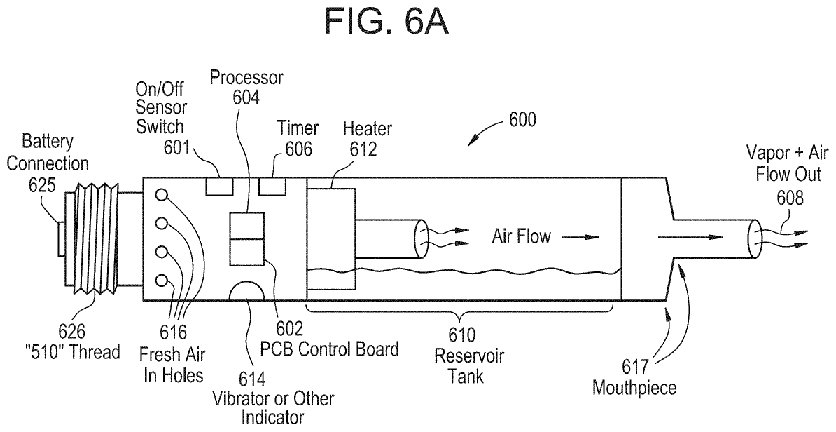

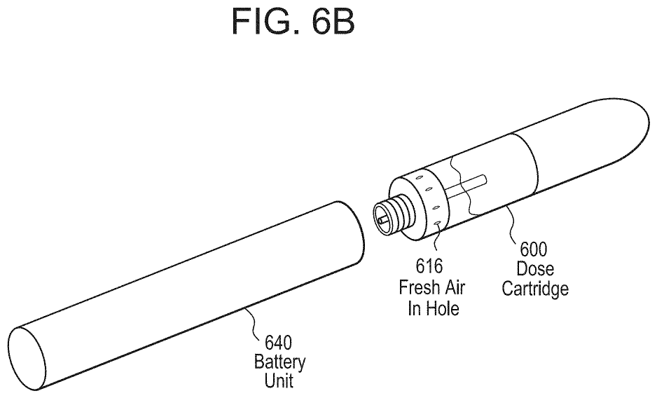

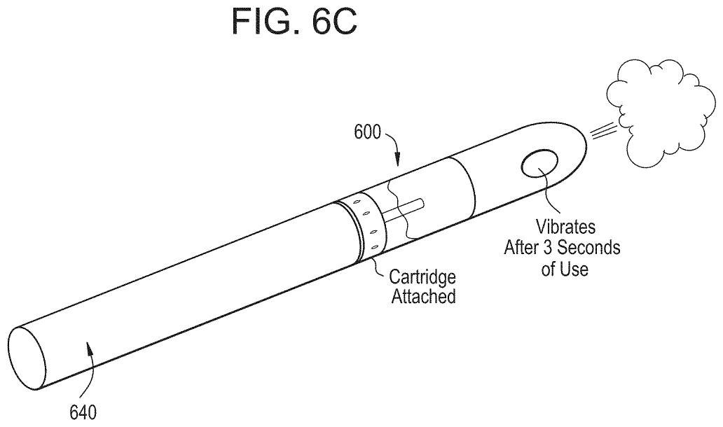

FIGS. 6A-6B illustrate an inhalation cartridge 600 (e.g., an inhalation device) according to another embodiment of the disclosure. The inhalation cartridge 600 includes a printed control board 602 having a processor 604 for controlling various operations and components of the inhalation cartridge 600. The PCB 602 can include a memory for storing data and a transmitter for transmitting data to external devices. The type of transmitter can take many forms, including; Bluetooth, Wi-Fi, cellular, near field radio, transponder and others. The processor 604 is configured to control various components including an on/off switch or sensor 601, a timer 606 and a heater 612. The inhalation cartridge 600 also includes an inlet 616, an outlet 608, a reservoir tank 610, a battery connection portion 625 (e.g., a battery connector), an indicator 614 and the mouthpiece 617 having channel extending to the outlet 608. Inhalation cartridge 600 may also incorporate various features and functions of the embodiments described above. For example, this cartridge can incorporate various sensors to turn on the heater 612 when inhalation by a user is detected, i.e., using pressure sensors, volume flow sensors or the like as described in the embodiments above. The inhalation device, through processing of dose amounts, can also provide indications to a user about the amount or dose that is cumulatively delivered.

While this embodiment shows the channel being formed in a mouthpiece 617, this portion may be embodied in the form of a nose inhalation device to permit the user to inhale the dose through their nasal passages. This can be particularly useful for administering decongestants and antihistamines to a user.

The reservoir tank 610 tank can be configured to refilling, either by the user or the manufacturer. The reservoir can be configured to accept and dispense liquid, and with non-liquid or semi-liquid substances; such as dried herbs, pastes, oils, powders, resins, butters, creams, and more.

In this embodiment, the cartridge is also configured to attached to a battery (not shown) using the battery connection portion 625 to provide power to the inhalation cartridge 600. The battery connection portion 625 shown in FIG. 6A has a standard 510 thread connection, but this may be configured as a standard 710 thread connection. This permits a user to easily interchange the battery with those complying with the 510 or 710 standard. However, the battery connection portion 625 may be configured with other thread counts or connecting methods. FIG. 6B shows the inhalation cartridge 600 where the dosing cartridge is separated from a battery unit 640. FIG. 6C shows the dosing cartridge connected with the battery unit. In this embodiment, the battery unit 640 has female thread connections configured to mate with, for example, the standard 510 or standard 710 connector of the dosing cartridge.

The inhalation cartridge 600 according to this embodiment is configured to use a processor 604 and a timer 606 to control the amount of time the heater 612 is activated when a user inhales through the cartridge. Specifically, after user begins to inhale through the device, a sensor (volumetric or pressure) as described in the embodiments above, detects the inhalation and the processor 604 starts the timer 606 and heater 612 to vaporize the substance from the reservoir tank 610. In this manner, the processor and timer function to accumulate the time duration over which the heater 612 is activated. When this time duration reaches a predetermined time limit, the processor 604 deactivates the heater 612 to end the vaporization of any further material from the reservoir tank 610. When the heater 612 is deactivated, the processor can also cause the indicator 614 to output a signal to the user that vaporization has ended. This output signal can be configured to provide indications to the user in accord with methods described with the embodiments described above, e.g., vibration, sound or light. The indicator 614 can also be configured as a dosage session indicator as described in the embodiments above.

This predetermined timing can be, for example, 3 seconds in duration based on some empirical evidence with known materials. However, this timing can vary depending on the substance to be vaporized and the amount/strength of drug, etc., contained in the substance.

Alternatively, the processor 604, instead of deactivating the heater 612, may continue operation of the heater 612 and cause the indicator 614 to provide an indication to the user that the desired dose has been delivered. Upon receiving this indication, the user can then cease inhalation to limit the amount of substance inhaled.

The predetermined time threshold may be set in advance, or alternatively, the predetermined time threshold may be variable based on considerations based on the initial temperature of the heater, the type of material to be vaporized in the reservoir, etc. As the materials containing the various substances may vaporize at different rates, the inhalation cartridge 600 may incorporate a switch or dial to enable a user to select a type of material, or an intended predetermined timing threshold, so that different doses of the same material, or the same dose of different materials can be administered with good precision.

The processor 604 can determine a total dosing amount based on accumulating the amount of doses delivered by the inhalation cartridge 600. In the case where a predetermined time threshold is used to deliver the dose amount, the processor 604 can update the accumulated dose amount and store this in the memory of the PCB 602. This dosage amount can be communicated externally via the transmitter of the PCB 602. Alternatively, each time a dose is delivered by the inhalation cartridge 600, an indication that a dose has been delivered may be transmitted to an external device to permit that device to track the total dose or usage.

Additionally, as the initial temperature of the heater may vary, it is also possible to modify the set predetermined limit may be adjusted to compensate for the time required for the heater to reach a vaporization temperature for the material in the reservoir. For example, if the heater of inhalation cartridge has been recently activated, the time to reach the vaporization temperature may be less then conditions where the inhalation device has been idle, and therefore, at a lower initial temperature. Thus, the processor 604 may be configured to adjust the predetermined limit by increasing the time of activation, or decreasing the time of activation to further ensure precision of the dosing amount.

While not shown, the inhalation cartridge 600 may incorporate a thermocouple to measure the temperature of the heater 612 to make adjustments to the predetermined time limit based on the current measure of the heater temperature. The processor 604 in combination with a thermocouple can effectively adjust for different external conditions, such as use in extremely cold or hot conditions. Therefore, the dosing precision can be further increased to permit proper dosing at temperatures below freezing and temperatures experienced within, for example, a sun heated automobile.

According to another variation, the inhalation cartridge 600 processor 604 may be configured to incorporate data from the timer and from the various sensors described in the embodiments detailed above to calculate the dosage information, i.e., by using the sensor 108 (optosensor) change and vapor intensity in combination with the timer information to calculate a dosage amount. Thus, the vapor intensity per unit time can be integrated over a time period to calculate dosage. Alternatively, the dosage can be determined based on an accumulation of time over which the heater is activated. This information may be stored in a memory or other storage unit associated with the inhalation device and/or transmitted to an external device.

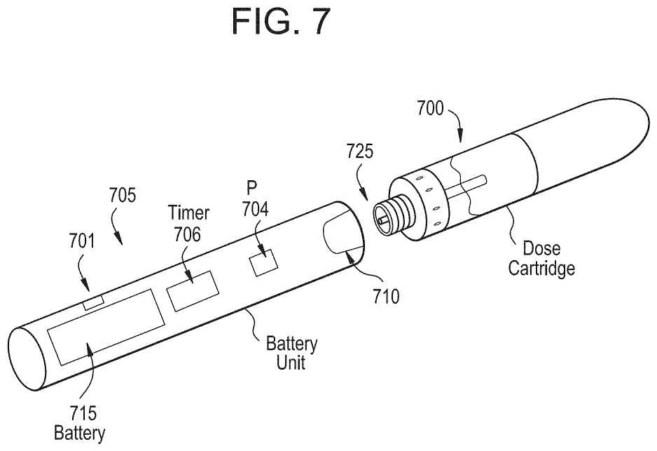

FIG. 7 illustrates a battery cartridge 705 (i.e., a battery unit) according to another embodiment of the disclosure. This battery cartridge 705 includes a battery 715, a timer 706 and a battery connection 725. This configuration can be used in combination with any inhalation device, e.g., an inhalation cartridge 700 that is connectable to the battery cartridge 705 via the battery connection 725. This battery connection may include a threaded connection, and this connection can be configured to comply with both the 510 standard or the 710 standard. Additionally, other battery connections can be used as well.

Under one configuration, the battery cartridge 705 can be configured such that the timer 706 determines when battery current is being drawn by an attached inhalation device to measure the time that a heater is activated in the inhalation device. The timer can include circuitry that cuts the power supplied to the inhalation device after heater current is drawn for a predetermined time to control a dosage amount similar to the embodiment of FIG. 6. The battery cartridge may also include a switch or dial 701 to permit a user to set the predetermined time used to control the dosage amount. Accordingly, any inhalation device can be converted, by use of this battery cartridge, into a device that can control a dosage amount of the inhalation device.

Additionally, the battery cartridge 705 may include a processor 704 to receive the user input from the switch 701 and the timer 706 to control the power delivered by the battery 715 via switches or the like. Further, this processor 704 can further include the processing capabilities of the embodiment of FIG. 6 to provide enhanced dosage control as described above.

In another embodiment, the inhalation devices described herein can be connected to a mobile device such as a smartphone or tablet and interfaced with a software application. The software application can record the doses that the user has inhaled and record the user's dosage experience. This information can be analyzed by the software to track and optimize the user's experience with the substance inhaled. To help improve analysis, the user could also enter personal information such as ailments, pains, weight and food intake. The information recorded can be used to accurately monitor a user's intake details and may be submitted to a doctor for review and/or improvement.

The application could also connect with other users via the internet. This could be used to share experiences, receive recommendations, and network with a community of users. The application may also be used as an ecommerce platform to purchase dosage capsules, or vaporizer equipment. The platform could offer specific substances based on a user's rated experience. Another enhanced use might be finding other users within geographic locations that may allow for social interactions and meetings. These enhanced services may be integrated with others over the internet.

The vaporizer device could also be locked by the user via the application. This could be used as a safety feature against undesired use (by children or others). There could be locking customizable lock setting to enhance safety or limit usage for those with low self-control.

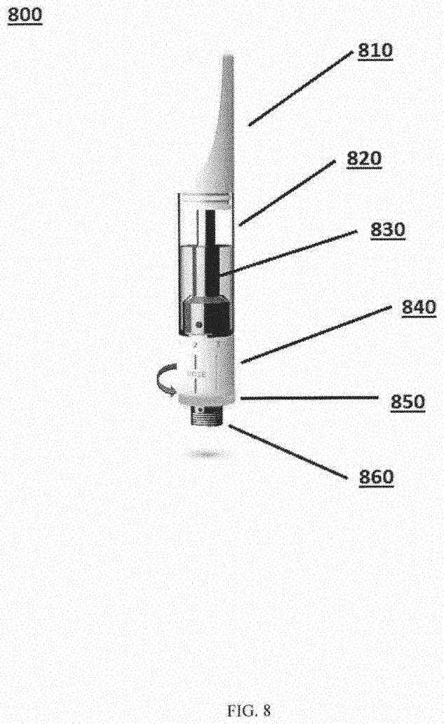

FIG. 8 is another diagram of an inhalation device. As shown in FIG. 8, inhalation device 800 may include a mouthpiece 810, a reservoir 820, an atomizer 830, a dose selection component 840, an output component 850, and a battery connector 860. The inhalation device 800 may include a controller 870, a memory 880, and a sensor 890 disposed within an outer housing of the inhalation device 800.

The mouthpiece 810 may be a component configured to permit a user to inhale vapor generated by the inhalation device 800. The reservoir 820 may be a component configured to store the substance to be vaporized. The atomizer 830 may be a component configured to vaporize the substance to generate vapor that is capable of being inhaled by the user via the mouthpiece 810.

The dose selection component 840 may a component configured to receive, via a user input, a dose selection of a substance to be vaporized. For example, the dose selection component 840 may be a dial, a touch screen display, a keyboard, a mouse, a button, a switch, a microphone, etc.

As shown in FIG. 8, the dose selection component 840 in this embodiment may be a generally cylindrically shaped dial that is circumferentially disposed on the inhalation device 800, and that is configured to rotate up to 360 degrees with respect to a longitudinal axis of the inhalation device 800. A circular dial would also be an alternate possibility.

The user may rotate the dial to input a dose selection, for example by actuating switches as the dial rotates. For example, the user may rotate the dial to align a predetermined portion of the dial with an indicator of a dose, or the dial itself may include a numerical indicator. In this way, the user may rotate the dial to input a dose selection. Alternatively, the user may rotate the dial to align an indicator of a dose on the dial with a predetermined portion of the inhalation device 800.

The dose selection component 840 may allow consumers to choose their desired intake amount. Consumers can use the dose selection component 840 to set a pre-set amount of vapor they wish to inhale. For example, the dose selection component 840 may be a dial that allows the consumer to choose between 1, 2, 3, etc. doses. A larger number of increments that the three indicated may also be used for finer selection, but for purposes of this description, the three selection dial will be used as the exemplar.

Each dose may represent an amount of vapor. Just as a non-limiting example, one dose can be preset to approximately 1 mg. By setting the device to 3 doses, the device will allow the consumer to inhale and consume 3 times the preset amount per dose, (i.e., a total of 3 milligrams of vapor corresponding to approximately 3 seconds of vapor production) and then will stop producing vapor.

In other implementations, the dose selection component 840 may expressly allow a consumer to select a target amount of the substance to be consumed. For example, the dose selection component 840 may allow the user to input a value of 5 milligrams, 10 milligrams, 12, milligrams, 15 milligrams, 19 milligrams, etc. of, e.g., nicotine equivalent, these values representing typical amounts contained in commonly available cigarette brands, and the dial may be labelled as such rather than by dose. Also, since the invention can be used with other inhalable medicants such as cannabis oil/THC and CBD, or antihistamines and other drugs, the dial may be alternately labeled with dose amounts suitable to the particular substance that is contained in the vaporizer.

The output component 850 may be a component configured to notify a user that a dose corresponding to a dose selection has been generated by the inhalation device 800. The output component 850 may be a display, a speaker, a haptic component, one or more lights inclusive of light emitting diodes (LEDs), etc. In this embodiment preferably a light is used.

The battery connector 860 may be a component configured to connect to a battery unit. The battery connector 860 may include a 510 standard thread, a magnetic connector, snap fit connector, a press fit connector, a friction based connector, a hardwired connector, etc.

The controller 870 may include a central processing unit (CPU), a graphics processing unit (GPU), an accelerated processing unit (APU), a microprocessor, and/or any processing component (e.g., a field-programmable gate array (FPGA), an application-specific integrated circuit (ASIC), etc.) that interprets and/or executes instructions. In some implementations, controller 870 may include one or more processors capable of being programmed to perform a function.

The memory 880 may include a random access memory (RAM), a read only memory (ROM), and/or another type of dynamic or static storage device (e.g., a flash memory, a magnetic memory, an optical memory, etc.) that stores information and/or instructions for use the controller 870.

The sensor 890 may include a sensor for sensing information (e.g., a global positioning system (GPS) component, a timer, an airflow sensor, a power sensor, a flow sensor, a temperature sensor, an accelerometer, a gyroscope, an actuator, etc.).

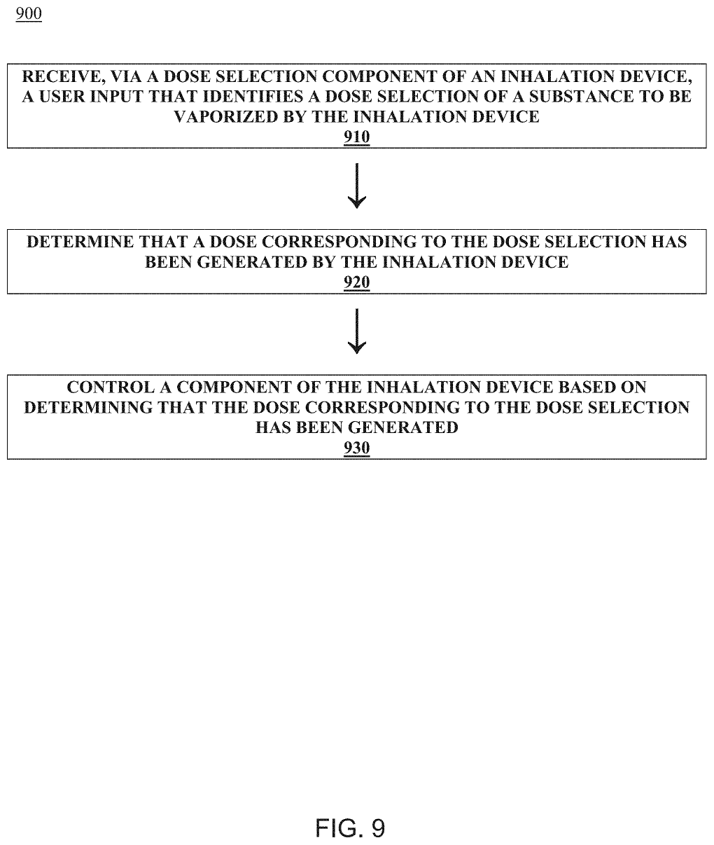

FIG. 9 is a diagram of a process for controlling a component of an inhalation device based on determining that a dose corresponding to a dose selection has been generated. This process can be used with the device of FIG. 8 but is not limited thereto.

As shown in FIG. 9, process 900 may include receiving, via a dose selection component of an inhalation device, a user input that identifies a dose selection of a substance to be vaporized by the inhalation device (block 910).

For example, the controller 870 of the inhalation device 800 may receive, via the dose selection component 840, a user input that identifies a dose selection of a substance to be vaporized by the inhalation device.

The dose selection component 840 may be a circumferential dial, and the user may rotate the dial to input a dose selection.

As further shown in FIG. 9, process 900 may include determining that a dose corresponding to the dose selection has been generated by the inhalation device (block 920).

For example, the controller 870 of the inhalation device 800 may determine that the inhalation device 800 has generated a dose corresponding to the dose selection. The controller 870 may determine that the dose has been generated, based on one or more sensors 890 of the inhalation device 800.

The controller 870 may determine that the dose has been generated based on an amount of time. For example, the controller 870 may determine an amount of time that the atomizer 830 has been operating (“on time”), and determine that the dose has been generated based on the on time.

The controller 870 may identify a start of the on time based on a user inhaling through the mouthpiece 810, based on the atomizer 830 turning on, based on power being delivered to the atomizer 830, based on detected air flow, etc.

The controller 870 may determine the on time of the heater/atomizer 830 based on the dose selection. For example, if the consumer selects 1 dose, then the on time may be 1.2 seconds. However, if the consumer selects 3 doses, then the on time may not be 3.6 seconds (i.e., 1.2 seconds×3). The first dose may require warm up time, and the atomizer 830 might not work as efficiently in the first dose as it does in the second dose or third dose. Accordingly, the on time may be non-linearly related to the number of doses, such as 1.2 seconds, 1.08 seconds and 1 second, for the three successive doses.

The controller may determine the on time based on the following equation:

On Time=Selected Number of Doses×(1+(Adjustments due to airflow+Adjustments due to power)).

For example, as shown by the above equation and in previously described embodiments, the controller 870 may determine the on time based on the dose selection, an airflow rate, and a power value that corresponds to an amount of power drawn from the battery. The controller 870 may determine an initial on time based on the dose selection, and may adjust the initial on time based on an airflow rate (e.g., detected by an airflow sensor) and/or a power value corresponding to an amount of power drawn from the battery (e.g., detected by a power sensor, a current sensor, a temperature sensor, an airflow sensor, etc.). Therefore, the on-time for the atomizer is essentially a different value for each “draw” (assuming a “draw” corresponds to a dose) on the vaporizer by the user, customized according to the sequence of each draw in the series of draws, airflow for each draw, and power drawn for each draw.

Airflow may come from an airflow sensor or an average estimate variable may be used. This variable may be predetermined from experience and/or empirical data from the testing of the hardware.

The amount of power drawn from the battery may be an average estimate, or may be predetermined from experience and/or empirical data from the testing of the hardware. Alternatively, the power value may vary depending on the number of doses selected by the consumer. If the consumer selects 3 doses, then more power may be used for the first dose to cause the atomizer 830 to obtain the desired temperature faster, as compared to the last dose when the temperature is already at or close to the desired value.

Power may be variable based on a measurement from an airflow sensor. For example, a high airflow rate may cool the atomizer 830, whereby more power is used to keep the atomizer at the desired temperature.

As further shown in FIG. 9, process 900 may include controlling a component of the inhalation device based on determining that the dose corresponding to the dose selection has been generated by the inhalation device (block 930).

For example, the controller 870 of the inhalation device 800 may control another component of the inhalation device 800 based on determining that the dose corresponding to the dose selection has been generated by the inhalation device 800. As an example, the controller 870 may control the atomizer 830 to turn off, based on determining that the dose has been generated. As another example, the controller 870 may control the output component 850 to output, for example, a visual notification that permits the user to identify that the dose has been generated.

The operations of the controller 870 described above in relation to FIG. 9 may be applied in relation to any of the other embodiments described herein. Further, any of the operations of the embodiments described herein may be applied to the embodiment of FIG. 8 and FIG. 9. In other words, any of the embodiments described herein may be modified by and/or used in conjunction with other embodiment(s).

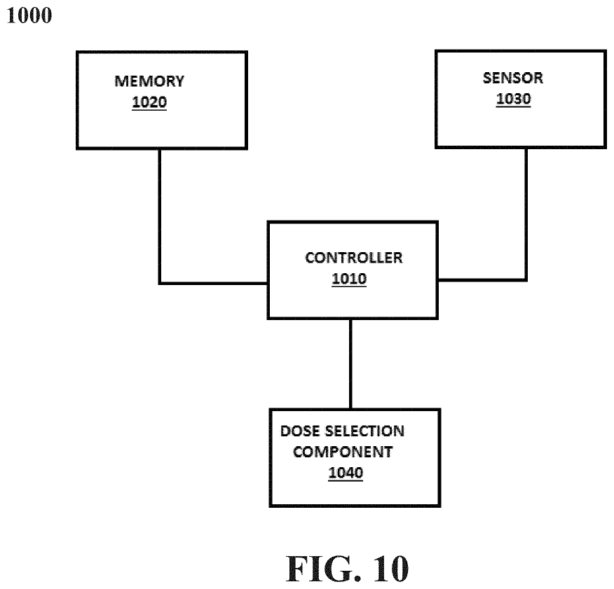

FIG. 10 is another diagram of an inhalation device. As shown in FIG. 10, inhalation device 1000 may include a controller 1010, a memory 1020, a sensor 1030, and a dose selection component 1040. The controller 1010, the memory 1020, the sensor 1030, and/or the dose selection component 1040 may be disposed within a main housing of the inhalation device 1000, or alternatively may be disposed within a battery unit configured to connect to the main housing of the inhalation device.

The controller 1010, memory 1020, sensor 1030, and dose selection component 1040 may be substantially the same as described above in connection with FIG. 8, or other embodiments as described herein.

That is, it should be understood that the inhalation device 1000 may include any number of separable components, such as a main housing (e.g., including a reservoir, atomizer, mouthpiece, etc.) and a battery unit that is configured to connect to the main housing (e.g., via a 510 connector, or the like). The controller 1010, memory 1020, sensor 1030, and dose selection component 1040 may be housed within either of the main housing or the battery unit. In any event, the controller 1010 may communicate with the memory 1020, sensor 1030, and dose selection component 1040 to perform, for example, operations described above in connection with FIG. 9.

In the event that the foregoing components are disposed in the battery unit, the controller 1010 may control the amount of power provided to an attached inhalation device in order to control a dosage amount. For example, the controller 1010 may receive a dose selection via the dose selection component 1040, and may control the amount of power provided to the inhalation device based on the dose selection.

As a particular example, the controller 1010 may determine when battery current is being drawn by an attached inhalation device to measure the time that a heater is activated in the inhalation device. The controller 1010 cut the power supplied to the inhalation device after heater current is drawn for a predetermined time, per dose, to control a total dosage amount similar to the embodiments of FIGS. 6, 8, and 9.

Accordingly, any inhalation device can be converted, by use of this battery cartridge or unit, into a device that can control a dosage amount of the inhalation device. The foregoing disclosure provides illustration and description, but is not intended to be exhaustive or to limit the implementations to the precise form disclosed. For example, although the specification focuses on a dose input selection unit in dial form, it will be understood that the capabilities of the invention could be obtained with other selection devices such as switches, a slider, a finger swipe, a level, a knob, a tab, or by blowing into or out of the device a set number of times corresponding to doses, or by shaking the device a set number of times according to doses. Modifications and variations are possible in light of the above disclosure or may be acquired from practice of the implementations.

As used herein, the term component is intended to be broadly construed as hardware, firmware, or a combination of hardware and software.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. An inhalation device comprising:
a dose selection component configured to receive, via a user input by a user, a dose selection of a substance to be vaporized by the inhalation device, in the form of a number of doses;
an airflow sensor configured to detect an airflow rate of the inhalation device; and
a controller configured to:
determine that a dose corresponding to the dose selection has been generated by the inhalation device; and
control components of the inhalation device based on determining that each dose corresponding to the selected number of doses has been generated by the inhalation device, comprising controlling a vaporizer of the inhalation device to cause the vaporizer to turn off after a determined or calculated time, which may be different for each dose of the number of doses; and controlling an output component of the inhalation device which permits the user to identify that the selected dose has been generated,
wherein the dose selection component is a circumferential dial configured to be rotatable by the user to permit the user to input the number of doses selected, and
wherein the controller is further configured to identify the airflow rate via the airflow sensor.

2. The inhalation device of claim 1, wherein the controller is further configured to:
determine an amount of time for the vaporizer of the inhalation device to be turned on for each dose, based on the number of doses selected, and
wherein the determining that each dose corresponding to the dose selection has been generated by the inhalation device comprises determining that the dose corresponding to the dose selection has been generated based on the amount of time for that dose.

3. The inhalation device of claim 2, wherein the controller is further configured to identify the airflow rate via the airflow sensor, and determine the amount of time by determining the amount of time based on the dose number selected and the airflow rate.

4. The inhalation device of claim 1, wherein the dose selection component is a button or a switch.

5. A method comprising:
receiving, via a dose selection component of an inhalation device, a user input that identifies a dose number selection of a substance to be vaporized by the inhalation device;
determining successively that a dose corresponding to each numbered dose selection has been generated by the inhalation device; and
controlling a vaporizer of the inhalation device to turn on when a user inhales and to turn off after a time individually determined for each successive dose of the number of doses, and
controlling an output component of the inhalation device which permits the user to identify that the selected dose has been generated,
wherein the dose selection component is a circumferential dial configured to be rotatable by the user to permit the user to input the number of doses selected, and
wherein the user input is effected by rotating the circumferential dial to one of a plurality of dose number settings.

6. The method of claim 5, further comprising:
determining an amount of time for the vaporizer of the inhalation device to be turned on based on the dose number selected, and
wherein the determining that the dose corresponding to the dose number selected has been generated by the inhalation device comprises determining that the on time determined for that numbered dose has been expended.

7. The method of claim 6, further comprising:
detecting an airflow rate of the inhalation device, and
wherein determining the amount of time comprises determining the amount of time based on the individual numbered dose and the airflow rate.

8. An inhalation device comprising:
a cylindrical rotatable dial encircling the inhalation device configured to rotate, via user manipulation, to input a target amount selection of a substance;
a light that encircles the inhalation device, and that is directly adjacent to the cylindrical rotatable dial;
an airflow sensor configured to detect an airflow rate of the inhalation device; and
a controller configured to:
determine that a target amount of the substance corresponding to the target amount selection has been generated by the inhalation device; and
control the light based on determining that the target amount corresponding to the target amount selection has been generated by the inhalation device, and
wherein the controller is further configured to identify the airflow rate via the airflow sensor.

9. The inhalation device of claim 8, wherein the substance is nicotine.

10. The inhalation device of claim 8, wherein the substance is tobacco.

11. The inhalation device of claim 8, wherein the target amount is in milligrams.

12. The inhalation device of claim 8, wherein the cylindrical rotatable dial is further configured to rotate, via user manipulation, to input a passcode that permits a vaporizer of the inhalation device to be turned on.
